(19) United States
(12) Patent Application Publication
(10) Pub. No.: US 2021/0077832 A1
(43) Pub. Date: Mar. 18, 2021

(54) METHODS OF TREATING CANCER WITH DENDRITIC CELL MOBILIZING AGENTS

(71) Applicants: Celldex Therapeutics, Inc., Hampton, NJ (US); Montefiore Medical Center, Bronx, NY (US); Albert Einstein College of Medicine, Bronx, NY (US)

(72) Inventors: Tibor KELER, Pipersville, PA (US); Michael YELLIN, Montclair, NJ (US); Chandan GUHA, Bronx, NY (US); Nitin OHRI, Bronx, NY (US)

(21) Appl. No.: 16/961,436

(22) PCT Filed: Jan. 25, 2019
(86) PCT No.: PCT/US2019/015215

§ 371 Date: Jul. 10, 2020

Related U.S. Application Data
(60) Provisional application No. 62/622,474, filed on Jan. 26, 2018.

(51) Int. Cl. A61N 5/10; A61P 35/00; C07K 16/28; A61K 38/17; A61K 45/06; A61K 39/395

(52) U.S. Cl. CPC A61N 5/1084; A61P 35/00; C07K 16/2827; C07K 16/2818; A61K 38/177; A61K 2039/507; A61K 45/06; A61K 39/3955; C07K 2317/21; C07K 2317/24; C07K 16/2875

(57) ABSTRACT

Methods of treating cancer comprising administering to patients a dendritic cell mobilizing agent (e.g., Flt3 ligand) in combination with radiation and/or immunoregulatory agents (e.g., checkpoint inhibitors), are disclosed.

### RELATED APPLICATIONS

This application claims the benefit of the priority date of U.S. Provisional Application No. 62/622,474, filed on Jan. 26, 2018, the content of which is hereby incorporated by reference in its entirety.

### GOVERNMENT FUNDING

This invention was made with Government support under Grant No. 5R44CA192435-03 awarded by the Small Business Innovation Research (SBIR) program. The Government has certain rights in the invention.

### SEQUENCE LISTING

The instant application contains a Sequence Listing which has been submitted electronically in ASCII format and is hereby incorporated by reference in its entirety. Said ASCII copy, created on Jan. 23, 2019, is named CDJ-398PC_SL.txt and is 6,709 bytes in size.

### BACKGROUND

Dendritic cells (DCs) are antigen-presenting cells which are known to process antigen material and present it on the cell surface to the T cells of the immune system. Once activated, they migrate to the lymph nodes where they interact with T cells and B cells and help shape the adaptive immune response. Thus dendritic cells, when presented with tumor specific antigens, play a critical role in the immune system's ability to target and kill tumor cells. However, dendritic cells are relatively rare cells in most tissues, and their functional capacity is often compromised in tumors and cancer patients.

Flt3 ligand (“Flt3-L″) is a cytokine that is known to stimulate the proliferation and differentiation of dendritic cells. Flt3-L binds to the Flt3 receptor expressed by progenitor cells leading to the development and expansion of multiple dendritic cell subtypes. Flt3-L has also been shown to inhibit tumor growth and stimulate tumor-specific immune responses.

It is an object of the present disclosure to provide improved methods for treating cancer patients with Flt3-L-based therapies.

### SUMMARY

Provided herein are methods for treating cancer comprising administering to a subject a dendritic cell mobilizing agent in combination with one or more immunoregulatory agents and/or radiation therapy.

In one aspect, the method comprises treating cancer in a subject in need thereof by simultaneously or sequentially administering an immunoregulatory agent, a dendritic cell mobilizing agent and radiation therapy. In another aspect, the method comprises treating cancer in a subject who has become or been determined to be resistant to an immunoregulatory agent, by simultaneously or sequentially administering a dendritic cell mobilizing agent and radiation therapy. In another aspect, the method comprises treating cancer in a subject by simultaneously or sequentially administering an immune checkpoint inhibitor and a Flt3 ligand (or nucleic acid coding therefor). In another aspect, the method comprises treating cancer in a subject who has become or been determined to be resistant to an immune checkpoint inhibitor, by administering a Flt3 ligand or nucleic acid coding therefor.

In one embodiment, the immunoregulatory agent is a checkpoint inhibitor. In another embodiment, the dendritic cell mobilizing agent is a Flt3 ligand or nucleic acid coding therefor. In certain embodiments, the method comprises treating cancer in a subject who is or has become resistant to an immune checkpoint inhibitor by simultaneously or sequentially administering a Flt3 ligand (or nucleic acid coding therefor) and radiation therapy.

In one embodiment, the immunoregulatory agent is administered either simultaneously or before or after administration of the radiation therapy. In another embodiment, the dendritic cell mobilizing agent may be administered simultaneously or before or after the radiation therapy.

In one embodiment, the Flt3 ligand is a soluble Flt3 ligand. In another embodiment, the soluble Flt3 ligand comprises the amino acid sequence of SEQ ID NO:4. In another embodiment, the immune checkpoint inhibitor includes an anti-PD1 antibody, an anti-PD-L1 antibody, or an anti-CTLA4 antibody. In another embodiment, the immune checkpoint inhibitor is nivolumab, pembrolizumab, atezolizumab, avelumab, durvalumab, tremelilumab or ipilimumab.

In one embodiment, radiation therapy is stereotactic body radiotherapy (SBRT).

In one embodiment, the cancer is leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

In certain embodiments, the cancer is lung cancer. In a particular embodiment, the cancer is non-small cell lung carcinoma.

In one embodiment, the Flt3 ligand or nucleic acid coding therefor is administered for 1-10 days. In another embodiment, the Flt3 ligand is administered for 1 to 10 days, beginning on the first day of radiotherapy. In another embodiment, the Flt3 ligand is administered for 5 days, beginning on the first day of radiotherapy. In another embodiment, the Flt3 ligand is administered at a dose of 10 to 200 ug/kg. In another embodiment, the Flt3 ligand is administered at a dose of 50 to 100 ug/kg. In another embodiment, the Flt3 ligand is administered at a dose of 75 ug/kg. In another embodiment, the Flt3 ligand is administered for 1 to 10 days beginning between five days before or after the first day of radiotherapy.

In one embodiment, the total radiation dose for a cycle of treatment is between 5 and 100 Gy. In another embodiment the radiation dose for a cycle of treatment is between 20 and 50 Gy on one occasion, between 10 and 30 Gy on each of two to four occasions, or between 5 and 20 Gy on each of 5 occasions. In another embodiment, the radiation dose for a cycle of treatment is 30 to 40 Gy on one occasion, 15 to 20 Gy on each of three occasions or 8 to 12 Gy on each of 5 occasions.

In one embodiment, the method further comprises a step of administering a dendritic cell activating agent. In certain embodiments, the dendritic cell activating agent is CD40L, an anti-CD40 antibody, a TLR activator or a STING activator. In another embodiment, the method further comprises administering an additional anti-cancer agent.

In another embodiment, the method comprises cycles of treatment at intervals of 2 to 5 months.

### BRIEF DESCRIPTION OF THE DRAWINGS

FIGS. 1A and 1B are tables showing the preliminary results for the first 9 patients treated with a soluble Flt3-L treatment and radiation therapy.

FIGS. 2A and 2B are graphs showing the overall survival and progression free survival of patients who received soluble Flt3-L treatment and radiation therapy.

FIG. 3 is a graph showing total glycolytic activity (TGA) of target lesions in patients who received soluble Flt3-L treatment and radiation therapy with and without prior immunotherapy treatment.

### DETAILED DESCRIPTION

As described herein, the invention is based in part on the discovery that dendritic cell mobilizing agents in combination with radiation therapy and/or immunoregulatory agents improve overall survival and progression free survival of cancer patients. Accordingly, the present disclosure provides methods for the treatment of cancer (e.g., solid tumors) comprising administering to a patient a dendritic cell mobilizing agent (e.g., Flt3-L) in combination radiation therapy and/or an immunoregulatory agent.

### I. Definitions

In order that the present description may be more readily understood, certain terms are first defined. Additional definitions are set forth throughout the detailed description. Unless defined otherwise, all technical and scientific terms used herein have the same meaning as commonly understood by one of ordinary skill in the art, and conventional methods of immunology, protein chemistry, biochemistry, recombinant DNA techniques, and pharmacology are employed.

As used herein, the terms “Flt3 ligand” and “Flt3-L” are used interchangeably and refer to a genus of polypeptides that bind and complex independently with Flt3 receptor found on progenitor and stem cells. The term “Flt3-L” encompasses proteins having the amino acid sequence 1 to 231 of SEQ ID NO:2 or the amino acid sequence 1 to 235 of SEQ ID NO:4, as well as those proteins having a high degree of similarity or a high degree of identity with the amino acid sequence 1 to 231 of SEQ ID NO:2 or the amino acid sequence 1 to 235 of SEQ ID NO:4, and which proteins are biologically active and bind the Flt3 receptor. In addition, the term refers to biologically active gene products of the DNA of SEQ ID NO:1 or SEQ ID NO:3. The term further refers to nucleic acids encoding any of the aforementioned Flt3-L polypeptides.

Further encompassed by the term “Flt3-L” are the membrane-bound proteins (which include an intracellular region, a membrane region, and an extracellular region), and soluble or truncated proteins which comprise primarily the extracellular portion of the protein, retain biological activity and are capable of being secreted. Specific examples of such soluble proteins are those comprising the sequence of amino acids 28-163 of SEQ ID NO:2 and amino acids 28-160 of SEQ ID NO:4.

Further encompassed by the term “Flt3-L” are variants thereof. A Flt3-L variant refers to a polypeptide substantially homologous to native Flt3-L, but which has an amino acid sequence different from that of native Flt3-L (human, murine or other mammalian species) because of one or more deletions, insertions or substitutions. The variant amino acid sequence preferably is at least 80% identical to a native Flt3-L amino acid sequence, most preferably at least 90% identical. The percent identity may be determined, for example, by comparing sequence information using the GAP computer program, version 6.0 described by Devereux et al. (Nucl. Acids Res. 12:387, 1984) and available from the University of Wisconsin Genetics Computer Group (UWGCG). The GAP program utilizes the alignment method of Needleman and Wunsch (J. Mol. Biol. 48:443, 1970), as revised by Smith and Waterman (Adv. Appl. Math 2:482, 1981). The preferred default parameters for the GAP program include: (1) a unary comparison matrix (containing a value of 1 for identities and 0 for non-identities) for nucleotides, and the weighted comparison matrix of Gribskoy and Burgess, Nucl. Acids Res. 14:6745, 1986, as described by Schwartz and Dayhoff, eds., Atlas of Protein Sequence and Structure, National Biomedical Research Foundation, pp. 353-358, 1979; (2) a penalty of 3.0 for each gap and an additional 0.10 penalty, for each symbol in each gap; and (3) no penalty for end gaps. Variants may comprise conservatively substituted sequences, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known. Naturally occurring Flt3-L variants can also be used in the methods described herein. Examples of such variants are proteins that result from alternate mRNA splicing events or from proteolytic cleavage of the Flt3-L protein, wherein the Flt3-L binding property is retained. Alternate splicing of MRNA may yield a truncated but biologically active Flt3-L protein, such as a naturally occurring soluble form of the protein, for example. Variations attributable to proteolysis include, for example, differences in the N- or C-termini upon expression in different types of host cells, due to proteolytic removal of one or more terminal amino acids from the Flt3-L protein (generally from 1-5 terminal amino acids).

The term “biologically active” as it refers to Flt3-L, means that the Flt3-L is capable of binding to Flt3. Alternatively, “biologically active” means the Flt3-L is capable of transducing a stimulatory signal to the cell through the membrane-bound Flt3.

The term “Isolated” means that the Flt3-L is free of association with other proteins or polypeptides, for example, as a purification product of recombinant host cell culture or as a purified extract.

The term “dendritic cell mobilizing agent” refers to an agent that increases the total number of circulating dendritic cells, as compared to the number of circulating dendritic cells prior to exposure to the mobilizing agent. A dendritic cell mobilizing agent may be any agent (e.g., small molecule, protein, or nucleic acid) that increases the number of circulating dendritic cells. For example, a dendritic cell mobilizing agent may be fms like tyrosine kinase 3 (Flt3-L), granulocyte-colony stimulating factor (G-CSF), granulocyte-macrophage colony-stimulating factor (GM-CSF), or variants thereof, and agonists of the receptors for these cytokines, such as progenipoietin (ProGP). An exemplary dendritic cell mobilizing agent is CDX-301 which is a soluble recombinant human Flt3-L.

The term “dendritic cell activating agent” refers to an agent that increases the activity of dendritic cells (e.g., antigen presentation, migration to lymph nodes, and interaction with T cells and B cells), as compared to the dendritic cell activity prior to exposure to the activating agent. Examples of a dendritic cell activating agents include, but are not limited to, CD40L, an anti-CD40 antibody, a TLR activator or a STING activator. Other suitable activating agents useful in the practice of the methods described herein include a RANKL peptide, TNF peptide, IL-1 peptide, CpG-rich DNA sequences, lipopolysaccharide (LPS), RIG1 helicase ligand, RNA, dsDNA or variations thereof (e.g., polypeptides or DNA sequences comprising one or more insertions, substitutions, or deletions).

The term “antibody” as used herein refers to polypeptides comprising at least one antibody derived antigen binding site (e.g., V_{H}/V_{L} region or Fv, or CDR), and includes whole antibodies and any antigen binding fragments (i.e., “antigen-binding portions” or “antigen binding fragments thereof”) or single chains thereof. Antibodies include known forms of antibodies. For example, the antibody can be a human antibody, a humanized antibody, a bispecific antibody, or a chimeric antibody. A “whole antibody” refers to a glycoprotein comprising at least two heavy (H) chains and two light (L) chains inter-connected by disulfide bonds, in which each heavy chain is comprised of a heavy chain variable region (abbreviated herein as V_{H}) and a heavy chain constant region; and each light chain is comprised of a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The terms “treat,” “treating,” and “treatment,” as used herein, refer to therapeutic measures described herein. The methods of treatment employ administration to a subject (such as a human) the combination disclosed herein in order to cure, delay, reduce the severity of, or ameliorate one or more symptoms of the disease or disorder or recurring disease or disorder, or in order to prolong the survival of a subject beyond that expected in the absence of such treatment.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding polypeptides, antibodies, or antibody fragments (e.g., V_{H}, V_{L}, CDR3), is intended to refer to a nucleic acid molecule in which the nucleotide sequences are essentially free of other genomic nucleotide sequences, e.g., those encoding other sequences may naturally flank the nucleic acid in human genomic DNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. The terms, “plasmid” and “vector” may be used interchangeably. However, other forms of expression vectors, such as viral vectors (e.g., replication defective retroviruses, adenoviruses and adeno-associated viruses), which serve equivalent functions are also contemplated.

The terms “treat,” “treating,” and “treatment,” as used herein, refer to therapeutic or preventative measures described herein. The methods of “treatment” employ administration to a subject with a tumor or cancer or a subject who is predisposed to having such a disease or disorder, a dendritic cell mobilizing agent (e.g., Flt3-L) as described herein, in order to prevent, cure, delay, reduce the severity of, or ameliorate one or more symptoms of the disease or disorder or recurring disease or disorder, or in order to prolong the survival of a subject beyond that expected in the absence of such treatment.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulvar cancer, thyroid cancer, hepatic carcinoma and various types of head and neck cancer.

As used herein, “solid tumor” refers to a subset of cancers characterized by abnormal mass of tissue which does not contain cysts or liquid areas. Examples include but are not limited to malignant sarcomas, carcinomas, and lymphomas.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

The term “therapeutic agent” in intended to encompass any and all compounds that have an ability to decrease or inhibit the severity of the symptoms of a disease or disorder, or increase the frequency and/or duration of symptom-free or symptom-reduced periods in a disease or disorder, or inhibit or prevent impairment or disability due to a disease or disorder affliction, or inhibit or delay progression of a disease or disorder, or inhibit or delay onset of a disease or disorder, or inhibit or prevent infection in an infectious disease or disorder. Non-limiting examples of therapeutic agents include small organic molecules, monoclonal antibodies, bispecific antibodies, recombinantly engineered biologics, RNAi compounds, and commercial antibodies.

As used herein, “administering” refers to the physical introduction of a composition comprising a therapeutic agent to a subject, using any of the various methods and delivery systems known to those skilled in the art. Exemplary routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “patient” includes human and other mammalian subjects that receive either prophylactic or therapeutic treatment.

The term “subject” includes any mammal. For example, the methods and compositions herein disclosed can be used to treat a subject having cancer. In a particular embodiment, the subject is a human.

The term “prophylaxis” refers to decreasing the likelihood of, or prevention of, a disease or condition (e.g., cancer, tumor burden, autoimmune disease, and allograft rejection).

The term “antigen” as used herein is defined as an entity which elicits an immune system response. The term herein may be abbreviated to “Ag.”

The term “immune cell” refers to cells that play a role in the immune response, including lymphocytes, such as B cells and T cells; natural killer cells; myeloid cells, such as monocytes, macrophages, eosinophils, mast cells, basophils, dendritic cells and granulocytes.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune response or reaction includes, e.g., activation or inhibition of a T cell, e.g., an effector T cell or a Th cell, such as a CD4⁺ or CD8⁺ T cell, or the inhibition of a Treg cell.

An “immunomodulator” or “immunoregulator” refers to an agent, that may be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

The terms “inhibitors” and “antagonists,” or “activators” and “agonists,” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a compound that reduces, blocks, or inactivates a constitutive activity.

The term “immune checkpoint inhibitor” refers to a group of molecules associated with signaling pathways in cells of the immune system which down-modulate or inhibit an immune response. Exemplary checkpoint inhibitors include, but are not limited to ipilimumab, nivolumab, pembrolizumab, durvalumab, and atezolizumab.

The term “inhibition” or “inhibit” as used herein, refers to any statistically significant decrease in biological activity, including partial and full blocking of the activity. For example, “inhibition” can refer to a statistically significant decrease of about 10%, 20%, 30%, 40%, 50%, 60%, 70%, 80%, 90%, 95%, 96%, 97%, 98%, 99%, or 100% in biological activity.

The term “inhibits growth” of a tumor includes any measurable decrease in the growth of a tumor, e.g., the inhibition of growth of a tumor by at least about 10%, for example, at least about 20%, at least about 30%, at least about 40%, at least about 50%, at least about 60%, at least about 70%, at least about 80%, at least about 90%, at least about 99%, or about 100%.

The term “radiotherapy”, or “radiation therapy”, means the treatment of cancer and other diseases with ionizing radiation. Exemplary types of radiation therapy include, stereotactic ablative radiotherapy (SABR)/stereotactic body radiation therapy (SBRT), or stereotactic radiosurgery (SRS).

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a”, “an” and “the” include plural referents unless the context clearly dictates otherwise. The use of “or” or “and” means “and/or” unless stated otherwise. Furthermore, use of the term “including” as well as other forms, such as “include”, “includes”, and “included”, is not limiting.

The term “about” as used herein when referring to a measurable value such as an amount, a temporal duration and the like, is encompasses variations of up to ±10% from the specified value. Unless otherwise indicated, all numbers expressing quantities of ingredients, properties such as molecular weight, reaction conditions, etc., used herein are to be understood as being modified by the term “about”.

### II. Dendritic Cell Mobilizing Agents

Dendritic cell mobilizing agents increase the total number of circulating dendritic cells and are useful in the methods of the invention. Suitable dendritic mobilizing agents for use in the methods described herein include, for example, Flt3-L, G-CSF, GM-CSF, IL-15 and agonists of the receptors for these cytokines, such as progenipoietin (ProGP) which is a dual receptor agonist of both the G-CSF and the Flt3 receptors (Fleming et al., Blood 94:49a (1999)). An exemplary dendritic cell mobilizing agent is Flt3-L, as described in U.S. Pat. Nos. 5,554,512, 5,843,423, and 6,632,424, the disclosures of which are incorporated by reference herein. For example, CDX-301 is a soluble recombinant human FMS-like tyrosine kinase-3 ligand (Flt3-L) that acts by uniquely binding FMS-like tyrosine kinase-3 (Flt3, CD135), which is expressed on hematopoietic stem cells (HSC), early progenitor cells, immature thymocytes, and steady state dendritic cells, resulting in the proliferation, differentiation, development and mobilization of these cells in the bone marrow, peripheral blood and lymphoid organs.

Other Flt3-L related dendritic cell mobilization agents suitable for use in methods described herein include those agents that bind Flt3 and transduce a signal. Such Flt3 binding proteins encompass agonistic antibodies that include monoclonal antibodies and humanized antibodies, and recombinantly-prepared agents that have at least one suitable antigen binding domain and are derived from agonistic antibodies that transduce Flt3 signaling.

In one embodiment, soluble Flt3-L is administered sequentially or simultaneously, with an immunoregulatory agent and/or radiation therapy. A soluble Flt3-L comprises all or part of the extracellular domain of a native Flt3-L but lacks the transmembrane region that would cause retention of the polypeptide on a cell membrane. A soluble Flt3-L advantageously comprises the native (or a heterologous) signal peptide when initially synthesized to promote secretion, but the signal peptide is cleaved upon secretion from the cell. Soluble Flt3-L retains the ability to bind the Flt3 receptor. Moreover, soluble Flt3-L may also include part of the transmembrane region or part of the cytoplasmic domain or other sequences, provided that the soluble Flt3-L protein can be secreted.

Soluble Flt3-L may be identified (and distinguished from its non-soluble membrane-bound counterparts) by separating intact cells which express the desired protein from the culture medium, e.g., by centrifugation, and assaying the medium (supernatant) for the presence of the desired protein. The presence of Flt3-L in the medium indicates that the protein was secreted from the cells and thus is a soluble form of the desired protein.

Soluble forms of Flt3-L possess many advantages over the native bound Flt3-L protein. Purification of the proteins from recombinant host cells is feasible, since the soluble proteins are secreted from the cells. Further, soluble proteins are generally more suitable for systemic administration (e.g., subcutaneous administration). Alternatively, other injectable routes may be suitable for the Flt3-L (e.g., intravenous).

Examples of soluble Flt3-L polypeptides include those comprising a substantial portion of the extracellular domain of a native Flt3-L protein. Such soluble mammalian Flt3-L proteins comprise amino acids 28 through 188 of SEQ ID NO:2 or amino acids 28 through 182—of SEQ ID NO:4. In addition, truncated soluble Flt3-L proteins comprising less than the entire extracellular domain can be used in the methods described herein. Such truncated soluble proteins are represented by the sequence of amino acids 28-163 of SEQ ID NO:2, and amino acids 28-160 of SEQ ID NO:4. When initially expressed within a host cell, soluble Flt3-L may additionally comprise one of the heterologous signal peptides described below that is functional within the host cells employed. Alternatively, the protein may comprise the native signal peptide, such that the mammalian Flt3-L comprises amino acids 1 through 188 of SEQ ID NO:2 or amino acids 1 through 182 of SEQ ID NO:4. In one embodiment, soluble Flt3-L can be expressed as a fusion protein comprising (from N- to C-terminus) a factor signal peptide, a FLAG® peptide described below and in U.S. Pat. No. 5,011,912, and soluble Flt3-L consisting of amino acids 28 to 188 of SEQ ID NO:2. This recombinant fusion protein can be expressed in and secreted from yeast cells. The FLAG® peptide facilitates purification of the protein, and subsequently may be cleaved from the soluble Flt3-L using bovine mucosal enterokinase. Isolated DNA sequences encoding soluble Flt3-L proteins may also be used in the methods described herein.

Truncated Flt3-L, including soluble polypeptides, may be prepared by any of a number of conventional techniques. A desired DNA sequence may be chemically synthesized using techniques known in the art. DNA fragments also may be produced by restriction endonuclease digestion of a full length cloned DNA sequence, and isolated by electrophoresis on agarose gels. Linkers containing restriction endonuclease cleavage site(s) may be employed to insert the desired DNA fragment into an expression vector, or the fragment may be digested at cleavage sites naturally present therein. The well-known polymerase chain reaction procedure also may be employed to amplify a DNA sequence encoding a desired protein fragment. As a further alternative, known mutagenesis techniques may be employed to insert a stop codon at a desired point, e.g., immediately downstream of the codon for the last amino acid of the extracellular domain.

In another approach, enzymatic treatment (e.g., using Bal 31 exonuclease) may be employed to delete terminal nucleotides from a DNA fragment to obtain a fragment having a particular desired terminus. Among the commercially available linkers are those that can be ligated to the blunt ends produced by Bal 31 digestion, and which contain restriction endonuclease cleavage site(s). Alternatively, oligonucleotides that reconstruct the N- or C-terminus of a DNA fragment to a desired point may be synthesized and ligated to the DNA fragment. The synthesized oligonucleotide may contain a restriction endonuclease cleavage site upstream of the desired coding sequence and position an initiation codon (ATG) at the N-terminus of the coding sequence.

Additionally isolated or homogeneous Flt3-L polypeptides, both recombinant and non-recombinant, may be used in the methods provided herein. Variants and derivatives of native Flt3-L proteins that retain the desired biological activity (e.g., the ability to bind Flt3) may be obtained by mutations of nucleotide sequences coding for native Flt3-L polypeptides. Alterations of the native amino acid sequence may be accomplished by any of a number of conventional methods. Mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site-specific mutagenesis procedures can be employed to provide an altered gene wherein predetermined codons can be altered by substitution, deletion or insertion. Exemplary methods of making the alterations set forth above are disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Kunkel (Proc. Natl. Acad. Sci. USA 82:488, 1985); Kunkel et al. (Methods in Enzymol. 154:367, 1987); and U.S. Pat. Nos. 4,518,584 and 4,737,462 all of which are incorporated by reference.

Flt3-L may be modified to create Flt3-L derivatives by forming covalent or aggregative conjugates with other chemical moieties, such as glycosyl groups, lipids, phosphate, acetyl groups and the like. Covalent derivatives of Flt3-L may be prepared by linking the chemical moieties to functional groups on Flt3-L amino acid side chains or at the N-terminus or C-terminus of a Flt3-L polypeptide or the extracellular domain thereof. Other derivatives of Flt3-L include covalent or aggregative conjugates of Flt3-L or its fragments with other proteins or polypeptides, such as by synthesis in recombinant culture as N-terminal or C-terminal fusions. For example, the conjugate may comprise a signal or leader polypeptide sequence (e.g. the α-factor leader of Saccharomyces) at the N-terminus of a Flt3-L polypeptide. The signal or leader peptide co-translationally or post-translationally directs transfer of the conjugate from its site of synthesis to a site inside or outside of the cell membrane or cell wall.

Flt3-L polypeptides described herein, include polypeptides with or without associated native-pattern glycosylation. Flt3-L expressed in yeast or mammalian expression systems (e.g., COS-7 cells) may be similar to or significantly different from a native Flt3-L polypeptide in molecular weight and glycosylation pattern, depending upon the choice of expression system and culture conditions. Expression of Flt3-L polypeptides in bacterial expression systems, such as E. coli, provides non-glycosylated molecules.

Equivalent DNA constructs that encode various additions or substitutions of amino acid residues or sequences, or deletions of terminal or internal residues or sequences not needed for biological activity or binding can be used in the methods described herein. For example, N-glycosylation sites in the Flt3-L extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. The murine and human Flt3-L proteins each comprise two such triplets, at amino acids 127-129 and 152-154 of SEQ ID NO:2, and at amino acids 126-128 and 150-152 of SEQ ID NO:4, respectively. Appropriate substitutions, additions or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Known procedures for inactivating N-glycosylation sites in proteins include those described in U.S. Pat. No. 5,071,972 and EP 276,846, are hereby incorporated by reference.

In another example, sequences encoding Cys residues that are not essential for biological activity can be altered to cause the Cys residues to be deleted or replaced with other amino acids, preventing formation of incorrect intramolecular disulfide bridges upon renaturation. Other equivalents are prepared by modification of adjacent dibasic amino acid residues to enhance expression in yeast systems in which KEX2 protease activity is present. EP 212,914 discloses the use of site-specific mutagenesis to inactivate KEX2 protease processing sites in a protein. KEX2 protease processing sites are inactivated by deleting, adding or substituting residues to alter Arg-Arg, Arg-Lys, and Lys-Arg pairs to eliminate the occurrence of these adjacent basic residues. Lys-Lys pairings are considerably less susceptible to KEX2 cleavage, and conversion of Arg-Lys or Lys-Arg to Lys-Lys represents a conservative and preferred approach to inactivating KEX2 sites. Both murine and human Flt3-L contain two KEX2 protease processing sites at amino acids 216-217 and 217-218 of SEQ ID NO:2 and at amino acids 211-212 and 212-213 of SEQ ID NO:4, respectively.

Nucleic acid sequences include isolated DNA and RNA sequences that hybridize to the native Flt3-L nucleotide sequences disclosed herein under conditions of moderate or severe stringency, and which encode biologically active Flt3-L. Conditions of moderate stringency, as defined by Sambrook et al. Molecular Cloning: A Laboratory Manual, 2 ed. Vol. 1, pp. 1.101-104, Cold Spring Harbor Laboratory Press, (1989), include use of a prewashing solution of 5×SSC, 0.5% SDS, 1.0 mM EDTA (pH 8.0) and hybridization conditions of about 55° C., 5×SSC, overnight. Conditions of severe stringency include higher temperatures of hybridization and washing. The skilled artisan will recognize that the temperature and wash solution salt concentration may be adjusted as necessary according to factors such as the length of the probe.

Due to the known degeneracy of the genetic code wherein more than one codon can encode the same amino acid, a DNA sequence may vary from that shown in SEQ ID NO:1 and SEQ ID NO:3 and still encode an Flt3-L protein having the amino acid sequence of SEQ ID NO:2 and SEQ ID NO:4, respectively. Such variant DNA sequences may result from silent mutations (e.g., occurring during PCR amplification), or may be the product of deliberate mutagenesis of a native sequence.

Isolated DNA sequences encoding biologically active Flt3-L, selected from: (a) DNA derived from the coding region of a native mammalian Flt3-L gene; (b) cDNA comprising the nucleotide sequence presented in SEQ ID NO:1 or SEQ ID NO:3; (c) DNA capable of hybridization to a DNA of (a) under moderately stringent conditions and which encodes biologically active Flt3-L; and (d) DNA which is degenerate as a result of the genetic code to a DNA defined in (a), (b) or (c) and which encodes biologically active Flt3-L. Flt3-L proteins encoded by such DNA equivalent sequences can be used in the methods described herein.

DNA that are equivalents to the DNA sequence of SEQ ID NO: 1 or SEQ ID NO:3, will hybridize under moderately stringent conditions to the native DNA sequence that encode polypeptides comprising amino acid sequences of 28-163 of SEQ ID NO:2 or 28-160 of SEQ ID NO:4. Examples of Flt3-L proteins encoded by such DNA, include, but are not limited to, Flt3-L fragments (soluble or membrane-bound) and Flt3-L proteins comprising inactivated N-glycosylation site(s), inactivated KEX2 protease processing site(s), or conservative amino acid substitution(s), as described above. Flt3-L proteins encoded by DNA derived from other mammalian species, wherein the DNA will hybridize to the cDNA of SEQ ID NO:1 or SEQ ID NO:3, can also be used in the methods described herein.

In certain embodiments, the nucleotide sequences of the dendritic mobilizing agents may be administered to a patient using gene therapy techniques known in the art. For example, the methods described herein can include administering to a subject a dendritic cell mobilizing agent (e.g., Flt3-L nucleotide sequence) using gene therapy in combination with one or more immunoregulatory agents and/or radiation therapy. In other embodiments, gene therapy methods known in the art can be used to transfect a cDNA encoding a dendritic cell mobilizing agent (e.g., Flt3-L) into delivery cells, which will transport the Flt3-L gene product to the targeted tissue.

In other embodiments, cells which have been treated with a dendritic cell mobilizing agent may be administered to a patient. For example, progenitor cells or dendritic cells (which have been expanded ex vivo and treated with a dendritic mobilizing agent) can be administered to a cancer patient (e.g., autologous transplantation) sequentially or simultaneously with one or more immunoregulatory agents and/or radiation therapy.

The term “autologous transplantation” is described in U.S. Pat. Nos. 5,199,942 and 6,632,424, which are incorporated herein by reference. Briefly, the term means a method for conducting autologous hematopoietic progenitor or stem cell transplantation, comprising: (1) collecting hematopoietic progenitor cells or stem cells from a patient prior to cytoreductive therapy; (2) expanding the hematopoietic progenitor cells or stem cells ex vivo with flt3-L to provide a cellular preparation comprising increased numbers of hematopoietic progenitor cells or stem cells; and (3) administering the cellular preparation to the patient in conjunction with or following cytoreductive therapy. Progenitor and stem cells may be obtained from peripheral blood harvest or bone marrow explants. Optionally, one or more cytokines, selected from the group listed above can be combined with flt3-L to aid in the proliferation of particular hematopoietic cell types or affect the cellular function of the resulting proliferated hematopoietic cell population. Of the foregoing, SF, IL-1, IL-3, EPO, G-CSF, GM-CSF and GM-CSF/IL-3 fusions are preferred, with G-CSF, GM-CSF and GM-CSF/IL-3 fusions being especially preferred. The term “allogeneic transplantation” means a method in which bone marrow or peripheral blood progenitor cells or stem cells are removed from a mammal and administered to a different mammal of the same species. The term “syngeneic transplantation” means the bone marrow transplantation between genetically identical mammals.

The transplantation method described above optionally may comprise a preliminary in vivo procedure comprising administering Flt3-L alone or in sequential or concurrent combination with a recruitment growth factor to a patient to recruit the hematopoietic cells into peripheral blood prior to their harvest. Suitable recruitment factors are listed above, and preferred recruitment factors are SF, IL-1 and IL-3.

The method described above optionally may comprise a subsequent in vivo procedure comprising administering Flt3-L alone or in sequential or concurrent combination with an engraftment growth factor to a patient following transplantation of the cellular preparation to facilitate engraftment and augment proliferation of engrafted hematopoietic progenitor or stem cells from the cellular preparation. Suitable engraftment factors are listed above, and the preferred engraftment factors are GM-CSF, G-CSF, IL-3, IL-1, EPO and GM-CSF/IL-3 fusions.

### III. Radiation Therapy

Ionizing radiation is widely used for the treatment of solid tumors. Several types of ionizing radiation can be used, including X-rays and gamma rays. Radiotherapy can be applied using a machine to focus the radiation on the tumor, or by placing radioactive implants directly into the tumor or in a nearby body cavity. Moreover, radiolabeled antibodies can be used to target tumor cells. Other radiotherapy techniques may also be used in the methods described herein, including intraoperative irradiation, particle beam radiation, as well as the use of radiosensitizers to make tumor cells more sensitive to radiation, or radioprotectants to protect normal cells.

In certain cases, the total irradiation dose can be spread over several sessions (i.e., dose fractionation) and can be spaced by at least 6 hours, days, or even weeks. Conventional definitive radiation treatment involves multiple treatments, generally 20-40, with low doses (<2-3 Gy) stretching over weeks.

High dose radiotherapy, e.g., >15-20 Gy, given in <=5 treatments also known as stereotactic ablative radiotherapy (SABR) or stereotactic body radiation therapy (SBRT) has been shown to provide therapeutic benefit to human patients with solid tumors. One type of SABR is stereotactic radiosurgery (SRS), which has been used for small intracranial tumors that was made possible by technology allowing for submillimeter delivery precision and steep dose gradients beyond the tumor target. SABR (or SBRT) has been developed for use on tumors outside of the brain and includes tumors of practically every major body site (e.g., lung tumors). Although radiotherapy has been shown to induce an abscopal effect in some instances (i.e., localized treatment of a tumor causes a shrinking of tumors outside the scope of the localized treatment, potentially due to an immunogenic response) (see E. Vacchelli et al., Oncoimmnology 5, e1214790 (2016)), high dose radiotherapy has also been shown to attenuate cancer cell immunogenicity (see C. Vanpouille-Box et al., Nat Commin 8, 15618 (2017)).

In general, radiation therapy, radio-immunotherapy or pre-targeted radioimmunotherapy are used for the treatment of diseases of oncological nature. “Radiotherapy”, or radiation therapy, means the treatment of cancer and other diseases with ionizing radiation. Ionizing radiation deposits energy that injures or destroys cells in the area being treated (the target tissue) by damaging their genetic material, making it impossible for these cells to continue to grow. Radiotherapy may be used to treat localized solid tumors, such as cancers of the skin, tongue, larynx, brain, breast, lung or uterine cervix. It can also be used to treat leukemia and lymphoma, i.e. cancers of the blood-forming cells and lymphatic system, respectively. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons are machines that produce x-rays of increasingly greater energy. The use of machines to focus radiation (such as x-rays) on a cancer site is called external beam radiotherapy.

Gamma rays are another form of photons used in radiotherapy. Gamma rays are produced spontaneously as certain elements (such as radium, uranium, and cobalt 60) release radiation as they decompose, or decay. Another technique for delivering radiation to cancer cells is to place radioactive implants directly in a tumor or body cavity. This is called internal radiotherapy. Brachytherapy, interstitial irradiation, and intracavitary irradiation are types of internal radiotherapy. In this treatment, the radiation dose is concentrated in a small area, and the patient stays in the hospital for a few days. Internal radiotherapy is frequently used for cancers of the tongue, uterus, and cervix. A further technique is intra-operative irradiation, in which a large dose of external radiation is directed at the tumor and surrounding tissue during surgery. Another approach is particle beam radiation therapy. This type of therapy differs from photon radiotherapy in that it involves the use of fast-moving subatomic particles to treat localized cancers. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation. Radio-sensitizers make the tumor cells more likely to be damaged, and radio-protectors protect normal tissues from the effects of radiation.

In one embodiment systemic radionuclides may be used.

In one aspect of the methods described herein, the radiation therapy uses ionizing radiation for treating cancer in a subject. In one embodiment, the radiation therapy is stereotactic ablative radiotherapy (SABR), stereotactic body radiation therapy (SBRT), or stereotactic radiosurgery (SRS). In a particular embodiment, the radiation therapy is stereotactic body radiation. In one embodiment, the total radiation dose for a cycle of treatment is between 5 and 100 Gy. In another embodiment, the total radiation dose for a cycle of treatment is between about 10 and about 100 Gy. In another embodiment, the total radiation dose for a cycle of treatment is between about 20 and about 100 Gy. In another embodiment, the total radiation dose for a cycle of treatment is between about 30 and about 100 Gy. In another embodiment, the total radiation dose for a cycle of treatment is between about 40 and about 100 Gy. In another embodiment, the total radiation dose for a cycle of treatment is between about 50 and about 100 Gy. In another embodiment, the total radiation dose for a cycle of treatment is between about 60 and about 100 Gy. In another embodiment, the total radiation dose for a cycle of treatment is between about 70 and about 100 Gy. In another embodiment, the total radiation dose for a cycle of treatment is between about 80 and about 100 Gy. In another embodiment, the total radiation dose for a cycle of treatment is between about 90 and about 100 Gy. In another embodiment, the total radiation dose for a cycle of treatment is about 100 Gy.

In one embodiment, the total radiation dose for a cycle of treatment is between about 20 to about 50 Gy. In one embodiment, the total radiation dose for a cycle of treatment is between about 20 to about 50 Gy on one occasion. In another embodiment, the total radiation dose for a cycle of treatment is between about 20 to about 50 Gy on each of two occasions.

In another embodiment, the total radiation dose for a cycle of treatment is between about 10 to about 30 Gy. In another embodiment, the total radiation dose for a cycle of treatment is between about 10 to about 30 Gy on one occasion. In another embodiment, the total radiation dose for a cycle of treatment is between about 10 to about 30 Gy on each of two occasions. In another embodiment, the total radiation dose for a cycle of treatment is between about 10 to about 30 Gy on each of three occasions. In another embodiment, the total radiation dose for a cycle of treatment is between about 10 to about 30 Gy on each of four occasions. In another embodiment, the total radiation dose for a cycle of treatment is between about 10 to about 30 Gy on each of five occasions. In another embodiment, the total radiation dose for a cycle of treatment is between about 10 to about 30 Gy on each of two to four occasions.

In another embodiment, the total radiation dose for a cycle of treatment is between about 5 and about 20 Gy. In another embodiment, the total radiation dose for a cycle of treatment is between about 5 and about 20 Gy on one occasion. In another embodiment, the total radiation dose for a cycle of treatment is between about 5 and about 20 Gy on each of two occasions. In another embodiment, the total radiation dose for a cycle of treatment is between about 5 and about 20 Gy on each of three occasions. In another embodiment, the total radiation dose for a cycle of treatment is between about 5 and about 20 Gy on each of four occasions. In another embodiment, the total radiation dose for a cycle of treatment is between about 5 and about 20 Gy on each of five occasions.

In a certain embodiments, the total radiation dose for a cycle of treatment is between about 20 and about 50 Gy on one occasion, between about 10 and about 30 Gy on each of two to four occasions, or between about 5 and about 20 Gy on each of 5 occasions.

In another embodiment, the total radiation dose for a cycle of treatment is between about 30 to about 40 Gy on one occasion. In another embodiment, the total radiation dose for a cycle of treatment is between about 30 to about 40 Gy on each of two occasions. In another embodiment, the total radiation dose for a cycle of treatment is between about 15 to about 20 Gy on one occasion. In another embodiment, the total radiation dose for a cycle of treatment is between about 15 to about 20 Gy on each of two occasions. In another embodiment, the total radiation dose for a cycle of treatment is between about 15 to about 20 Gy on each of three occasions. In another embodiment, the total radiation dose for a cycle of treatment is between about 15 to about 20 Gy on each of four occasions. In another embodiment, the total radiation dose for a cycle of treatment is between about 8 to about 12 Gy on one occasion. In another embodiment, the total radiation dose for a cycle of treatment is between about 8 to about 12 Gy on each of two occasions. In another embodiment, the total radiation dose for a cycle of treatment is between about 8 to about 12 Gy on each of three occasions. In another embodiment, the total radiation dose for a cycle of treatment is between about 8 to about 12 Gy on each of four occasions. In another embodiment, the total radiation dose for a cycle of treatment is between about 8 to about 12 Gy on each of five occasions. In another embodiment, the total radiation dose for a cycle of treatment is between about 8 to about 12 Gy on each of six occasions.

In a certain embodiments, the total radiation dose for a cycle of treatment is between about 30 to about 40 Gy on one occasion, about 15 to about 20 Gy on each of three occasions, or about 8 to about 12 Gy on each of 5 occasions.

### IV. Immunoregulatory Agents

Immunoregulatory agents include small molecule drugs, antibodies or antigen binding portions thereof, and/or protein ligands that are effective in stimulating immune responses to thereby further enhance, stimulate or upregulate immune responses in a patient.

In one embodiment, the immunoregulatory agent is (i) an agonist of a stimulatory (e.g., co-stimulatory) molecule (e.g., receptor or ligand) and/or (ii) an antagonist of an inhibitory signal or molecule (e.g., receptor or ligand) on immune cells, such as T cells. In either case, the agonistic or antagonistic molecule results in amplifying immune responses, such as antigen-specific T cell responses. For example, collectively, these molecules may be called immunoregulatory agents. In certain aspects, an immunoregulatory agent is enhances innate immunity, e.g., by acting as (i) an agonist of a stimulatory (including a co-stimulatory) molecule (e.g., receptor or ligand) or (ii) an antagonist of an inhibitory (including a co-inhibitory) molecule (e.g., receptor or ligand) on cells involved in innate immunity, e.g., NK cells.

As described above, T cell responses can be stimulated by administering an antagonist (inhibitor or blocking agent) of a protein that inhibits T cell activation. Such inhibitors are often called immune checkpoint inhibitors. For example, potential targets for checkpoint inhibitors include CTLA-4, PD-1, PD-L1, PD-L2, and LAG-3, and any of the following proteins: TIM-3, Galectin 9, CEACAM-1, BTLA, CD69, Galectin-1, TIGIT, CD113, GPR56, VISTA, B7-H3, B7-H4, 2B4, CD48, GARP, PD1H, LAIR1, TIM-1, and TIM-4. Exemplary immune checkpoint inhibitors include Opdivo™ (nivolumab/BMS-936558) (to PD-1), Yervoy™ (ipilimumab) or Tremelimumab (to CTLA-4), Tecentriq™ (atezolizmab) (to PD-L1), Durvalumab (to PD-L1), Bavencio™ (Avelumab) (to PD-L1), and Pembrolizumab/MK-3475 (to PD-1).

Alternatively, T cell responses can be stimulated by administering an agonist of a protein that stimulates T cell activation, such as B7-1, B7-2, CD28, 4-1BB (CD137), 4-1BBL, ICOS, ICOS-L, OX40, OX40L, CD70, CD27, CD40, DR3 and CD28H.

Other targets for immunoregulation include members of the immunoglobulin super family (IgSF). For example, dendritic cell mobilizing agent, e.g., described herein, may be administered to a subject with an agent that targets a member of the IgSF family to increase an immune response. For example, a dendritic cell mobilizing agent may be administered with an agent that targets (or binds specifically to) a member of the B7 family of membrane-bound ligands or a member of the TNF and TNFR family of molecules (ligands or receptors). For example, members of the B7 family of molecules may include, but is not limited to, B7-1, B7-2, B7-H1 (PD-L1), B7-DC (PD-L2), B7-H2 (ICOS-L), B7-H3, B7-H4, B7-H5 (VISTA), and B7-H6 or a co-stimulatory or co-inhibitory receptor binding specifically to a B7 family member. Examples of the TNF and TNFR family of molecules (ligands or receptors) may include, but is not limited to, CD40 and CD40L, OX-40, OX-40L, CD70, CD27L, CD30, CD30L, 4-1BBL, CD137, TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TALI, APRIL, BCMA, LTβR, LIGHT, DcR3, HVEM, VEGI/TL1A, TRAMP/DR3, EDA1, EDA2, TNFR1, Lymphotoxin α/TNFβ, TNFR2, TNFα, LTβR, Lymphotoxin α 1β2, FAS, FASL, RELT, DR6, TROY, and NGFR (see, e.g., Tansey (2009) Drug Discovery Today 00:1).

Other exemplary agents that modulate one of the above proteins and may be combined with dendritic cell mobilizing agents, e.g., those described herein, for treating cancer (e.g., lung cancer), include: galiximab (to B7.1), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TALI), CP-870893 (to CD40), Lucatumumab (to CD40), Dacetuzumab (to CD40), Muromonab-CD3 (to CD3), or pidilizumab (to PD-1).

Other molecules that can be combined with dendritic cell mobilizing agents for the treatment of cancer include antagonists of inhibitory receptors on NK cells or agonists of activating receptors on NK cells, e.g., an antagonists of KIR (e.g., lirilumab).

T cell activation is also regulated by soluble cytokines, and dendritic cell mobilizing agents may be administered to a subject, e.g., having cancer, with antagonists of cytokines that inhibit T cell activation or agonists of cytokines that stimulate T cell activation.

In another embodiment, dendritic cell mobilizing agents can be used in combination with (i) antagonists (or inhibitors or blocking agents) of proteins of the IgSF family or B7 family or the TNF family that inhibit T cell activation or antagonists of cytokines that inhibit T cell activation (e.g., IL-6, IL-10, TGF-β, VEGF; “immunosuppressive cytokines”) and/or (ii) agonists of stimulatory receptors of the IgSF family, B7 family or the TNF family or of cytokines that stimulate T cell activation, for stimulating an immune response, e.g., for treating proliferative diseases, such as cancer.

Other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

Dendritic cell mobilizing agents may also be administered with agents that inhibit TGF-β signaling.

Additional agents that may be combined with a dendritic cell mobilizing agent include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines, bleomycin, bortezomib, cyclophosphamide, doxorubicin, idarubicin, mitoxantrone, epirubicin, oxaliplatin, patupilone, septacidin, shikonin, vorinostat, wogonin, and paclitaxel (see e.g., Garg A D, Int J Dev Biol (2015) 59: 131).

Other therapies that may be combined with a dendritic cell mobilizing agent include therapies that deplete or block Treg cells, e.g., an agent that specifically binds to CD25.

Another therapy that may be combined with a dendritic cell mobilizing agent is a therapy that inhibits a metabolic enzyme such as indoleamine dioxigenase (IDO), dioxigenase, arginase, or nitric oxide synthetase.

Another class of agents that may be used with a dendritic cell mobilizing agent includes agents that inhibit the formation of adenosine or inhibit the adenosine A2A receptor.

Other therapies that may be combined with a dendritic cell mobilizing agent for treating cancer include therapies that reverse/prevent T cell anergy or exhaustion and therapies that trigger an innate immune activation and/or inflammation at a tumor site.

A dendritic cell mobilizing agent may be combined with more than one immunoregulatory agents, and may be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells; a therapy that stimulates positive immune regulation, e.g., with agonists that stimulate the CD-137, OX-40, and/or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno-suppressive cytokines.

Dendritic cell mobilizing agents described herein can be used together with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites.

In certain embodiments, the nucleotide sequences of any of the above mentioned immunoregulatory agents may be administered to a patient using gene therapy techniques known in the art.

### V. Additional Anti-Cancer Agents

The combination therapies described herein may also be used in conjunction with other therapies (i.e., anti-cancer therapies).

For example, the therapies described herein can be used in combination (e.g., simultaneously or separately) with one or more standard treatments, such as chemotherapy (e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, or camptothecin+apo21/TRAIL (a 6× combo)), one or more proteasome inhibitors (e.g., bortezomib or MG132), one or more Bcl-2 inhibitors (e.g., BH3I-2′ (bcl-xl inhibitor), indoleamine dioxygenase-1 inhibitor (e.g., INCB24360, indoximod, NLG-919, or F001287), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), anti-angiogenic agents targeting VEGFR2 (e.g., Cyramza™/ramucirumab), synthetic triterpenoids (see Hyer et al., Cancer Research 2005; 65:4799-808), c-FLIP (cellular FLICE-inhibitory protein) modulators (e.g., natural and synthetic ligands of PPARy (peroxisome proliferator-activated receptor y), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK3β inhibitors, IAP inhibitors and/or genotoxic drugs.

The dendritic cell mobilizing agents and combination therapies described herein can further be used in combination with one or more anti-proliferative cytotoxic agents. Classes of compounds that may be used as anti-proliferative cytotoxic agents include, but are not limited to, the following:

Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN™) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with dendritic cell mobilizing agents include, without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone Bl, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with the dendritic cell mobilizing agents described herein, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX™, can also be administered to the patient. When employing the methods or compositions described herein, other agents used in the modulation of tumor growth or metastasis in a clinical setting, such as antimimetics, can also be administered as desired.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

### VI. Patient Populations

The methods provided herein can be used to treat a broad variety of cancers, including, but not limited to, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system. In a particular embodiment, the cancer is lung cancer (e.g., NSCLC).

In one embodiment, the patient has evidence of recurrent or persistent disease following primary chemotherapy. In another embodiment, the patient has had at least one prior platinum based chemotherapy regimen for management of primary or recurrent disease. In another embodiment, the patient has a cancer that is platinum-resistant or refractory. In another embodiment, the patient has evidence of recurrent or persistent disease following a) primary treatment or b) an adjuvant treatment. In another embodiment, the patient has a cancer that has become or been determined to be resistant to an immunoregulatory agent. For example, the patient has evidence of recurrent or persistent disease following treatment with a checkpoint inhibitor (e.g., ipilimumab, nivolumab, pembrolizumab, durvalumab, or atezolizumab).

In another embodiment, the patient has an advanced cancer. In one embodiment, the term “advanced” cancer denotes a cancer above Stage II. In another, “advanced” refers to a stage of disease where chemotherapy is typically recommended, which is any one of the following: 1. in the setting of recurrent disease: any stage or grade; 2. stage IC or higher, any grade; 3. stage IA or IB, grade 2 or 3; or 4. in the setting of incomplete surgery or suspected residual disease after surgery (where further surgery cannot be performed): any stage or grade.

### VII. Treatment Protocols

Dendritic cell mobilizing agents can be administered prior to, sequentially, or simultaneously with one or more immunoregulatory agents and/or radiation therapy.

In one embodiment, administration of the dendritic cell mobilizing agent and the immunoregulatory agent (e.g., checkpoint inhibitor) starts at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the dendritic cell mobilizing agent has been administered.

In another embodiment, the administration of the dendritic cell mobilizing agent and radiation start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the radiation may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the dendritic cell mobilizing agent has been administered.

In certain embodiments, the dendritic cell mobilizing agent and the immunoregulatory agent, are administered simultaneously, e.g., are infused simultaneously, e.g., over a period of 30 or 60 minutes, to a patient. Alternatively, the dendritic cell mobilizing agent may be co-formulated with the immunoregulatory agent.

In another embodiment, the methods described herein comprise treatment intervals (cycles) of 1 to 6 months. In some embodiments, a cycle of treatment is an interval of 1 to 6 months, 1 to 5 months, 1 to 4 months, 1 to 3 months, or 1 to 2 months. In other embodiments, a cycle of treatment is 6 months, 5 months, 4 months, 3 months, 2 months, or 1 month.

In one embodiment, the dendritic cell mobilizing agent is administered for 1-10 days during a treatment cycle. In some embodiments, the dendritic cell mobilizing agent is administered for 1-9 days, 1-8 days, 1-7 days, 1-6 days, 1-5 days, 1-4 days, 1-3 days, or 1-2 days during the treatment cycle. In another embodiment, the dendritic cell mobilizing agent is administered for 1 day, 2 days, 3 days, 4 days, 5 days, 6 days, 7 days 8 days, 9 days or 10 day during the treatment cycle.

In one embodiment, the dendritic cell mobilizing agent is administered within 5 days before or after the first day of radiation therapy. In another embodiment, the dendritic cell mobilizing agent is administered within 4 days before or after the first day of radiation therapy. In another embodiment, the dendritic cell mobilizing agent is administered within 3 days before or after the first day of radiation therapy. In another embodiment, the dendritic cell mobilizing agent is administered within 2 days before or after the first day of radiation therapy. In another embodiment, the dendritic cell mobilizing agent is administered within 1 day before or after the first day of radiation therapy.

In one embodiment, the dendritic cell mobilizing agent is administered beginning on the first day of radiation therapy. In certain embodiments, the dendritic cell mobilizing agent is administered for 5 days beginning on the first day of radiation therapy. In certain embodiments, the dendritic cell mobilizing agent is administered for 4 days beginning on the first day of radiation therapy. In certain embodiments, the dendritic cell mobilizing agent is administered for 3 days beginning on the first day of radiation therapy. In certain embodiments, the dendritic cell mobilizing agent is administered for 2 days beginning on the first day of radiation therapy. In certain embodiments, the dendritic cell mobilizing agent is administered for 1 day beginning on the first day of radiation therapy.

In certain embodiments, the immunoregulatory agent is an immune checkpoint inhibitor. In one embodiment the immune checkpoint inhibitor is an anti-PD1 antibody. In another embodiment the immune checkpoint inhibitor is an anti-PD-L1 antibody. In another embodiment the immune checkpoint inhibitor is an anti-CTLA4 antibody. In another embodiment, the immune checkpoint inhibitor is selected from the group consisting of nivolumab, pembrolizumab, atezolizumab, avelumab, durvalumab, tremelilumab and ipilimumab. In another embodiment, the immune checkpoint inhibitor is nivolumab. In another embodiment, the immune checkpoint inhibitor is pembrolizumab. In another embodiment, the immune checkpoint inhibitor is atezolizumab. In another embodiment, the immune checkpoint inhibitor is avelumab. In another embodiment, the immune checkpoint inhibitor is durvalumab. In another embodiment, the immune checkpoint inhibitor is tremelilumab. In another embodiment, the immune checkpoint inhibitor is ipilimumab.

In another embodiment, the dendritic cell mobilizing agent is selected from the group consisting of FLT3-L, G-CSF, and GM-CSF. In another embodiment, the dendritic cell mobilizing agent is an agonist of any one of the receptors for FLT3-L, G-CSF, and GM-CSF. In another embodiment, the agonist is progenipoietin (ProGP). In another embodiment, the mobilizing agent is interleukin-15 (IL-15).

In certain embodiments, the dendritic cell mobilizing agent is a Flt3 ligand (Flt3-L) polypeptide or variant thereof. In another embodiment, the Flt3-L polypeptide comprises an amino acid sequence as shown in SEQ ID NO. 4. In another embodiment, the polypeptide comprises the soluble portion of the extracellular domain of a native Flt3-L protein. For example, the Flt3-L polypeptide may comprise the amino acids 28 through 182 of SEQ ID NO:4. In addition, truncated soluble Flt3-L proteins comprising less than the entire extracellular domain are also useful in the methods describe herein. For example, such truncated soluble proteins may be represented by amino acids 28-160 of SEQ ID NO:4. Isolated DNA sequences encoding soluble Flt3-L proteins may also be used in the methods described herein.

In one embodiment, the Flt3-L is administered to the patient at a dose of about 10 to about 200 ug/kg. In another embodiment, the Flt3-L is administered to the patient at a dose of about 20 to about 200 ug/kg. In another embodiment, the Flt3-L is administered to the patient at a dose of about 30 to about 200 ug/kg. In another embodiment, the Flt3-L is administered to the patient at a dose of about 40 to about 200 ug/kg. In another embodiment, the Flt3-L is administered to the patient at a dose of about 50 to about 200 ug/kg. In another embodiment, the Flt3-L, is administered to the patient at a dose of about 60 to about 200 ug/kg. In another embodiment, the Flt3-L is administered to the patient at a dose of about 70 to about 200 ug/kg. In another embodiment, the Flt3-L is administered to the patient at a dose of about 80 to about 200 ug/kg. In another embodiment, the Flt3-L is administered to the patient at a dose of about 90 to about 200 ug/kg. In another embodiment, the Flt3-L is administered to the patient at a dose of about 100 to about 200 ug/kg. In another embodiment, the Flt3-L is administered to the patient at a dose of about 110 to about 200 ug/kg. In another embodiment, the Flt3-L is administered to the patient at a dose of about 120 to about 200 ug/kg. In another embodiment, the Flt3-L is administered to the patient at a dose of about 130 to about 200 ug/kg. In another embodiment, the Flt3-L is administered to the patient at a dose of about 140 to about 200 ug/kg. In another embodiment, the Flt3-L is administered to the patient at a dose of about 150 to about 200 ug/kg. In another embodiment, the Flt3-L is administered to the patient at a dose of about 160 to about 200 ug/kg. In another embodiment, the Flt3-L is administered to the patient at a dose of about 170 to about 200 ug/kg. In another embodiment, the Flt3-L is administered to the patient at a dose of about 180 to about 200 ug/kg. In another embodiment, the Flt3-L is administered to the patient at a dose of about 190 to about 200 ug/kg. In another embodiment, the Flt3-L is administered to the patient at a dose of about 200 ug/kg.

In another embodiment, the Flt3-L is administered to the patient at a dose of about 50 to about 100 ug/kg. In another embodiment, the Flt3-L is administered to the patient at a dose of about 60 to about 100 ug/kg. In another embodiment, the Flt3-L is administered to the patient at a dose of about 70 to about 100 ug/kg. In another embodiment, the Flt3-L is administered to the patient at a dose of about 80 to about 100 ug/kg. In another embodiment, the Flt3-L is administered to the patient at a dose of about 90 to about 100 ug/kg.

In another embodiment, the Flt3-L is administered to the patient at a dose of about 50 ug/kg. In another embodiment, the Flt3-L is administered to the patient at a dose of about 55 ug/kg. In another embodiment, the Flt3-L is administered to the patient at a dose of about 60 ug/kg. In another embodiment, the Flt3-L is administered to the patient at a dose of about 65 ug/kg. In another embodiment, the Flt3-L is administered to the patient at a dose of about 70 ug/kg. In another embodiment, the Flt3-L is administered to the patient at a dose of about 75 ug/kg. In another embodiment, the Flt3-L is administered to the patient at a dose of about 80 ug/kg. In another embodiment, the Flt3-L is administered to the patient at a dose of about 85 ug/kg. In another embodiment, the Flt3-L is administered to the patient at a dose of about 90 ug/kg. In another embodiment, the Flt3-L is administered to the patient at a dose of about 95 ug/kg. In another embodiment, the Flt3-L is administered to the patient at a dose of about 100 ug/kg.

In another embodiment, the Flt3-L is administered is administered intravenously. In another embodiment, the Flt3-L is administered subcutaneously.

### VIII. Outcomes

The efficacy of the treatment methods provided herein can be assessed using any suitable means. In one embodiment, the treatment produces at least one therapeutic effect selected from the group consisting of reduction in growth rate of tumor, reduction in size of tumor, reduction in number of metastatic lesions over time, increase in duration of progression-free survival, and increase in overall response rate. The method provided herein, may inhibit tumor growth by at least about 10%, for example, at least about 20%, at least about 30%, at least about 40%, at least about 50%, at least about 60%, at least about 70%, at least about 80%, at least about 90%, at least about 99%, or 100%.

In one embodiment, the treatment methods described herein result in greater than 50% cancer cell death. In another embodiment, the treatment results in greater than 60% cancer cell death, greater than 65% cancer cell death, greater than 70% cancer cell death, greater than 75% cancer cell death, greater than 80% cancer cell death, greater than 85% cancer cell death, greater than 90% cancer cell death, greater than 95% cancer cell death, or 100% cancer cell death.

With respect to target lesions, responses to therapy may include:

Complete Response (CR): Disappearance of all target lesions. Any pathological lymph nodes (whether target or non-target) must have reduction in short axis to <10 mm;

Partial Response (PR): At least a 30% decrease in the sum of the diameters of target lesions, taking as reference the baseline sum diameters;

Progressive Disease (PD): At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression); and

Stable Disease (SD): Neither sufficient shrinkage to qualify for PR, nor sufficient increase to qualify for PD, taking as reference the smallest sum diameters while on study. (Note: a change of 20% or less that does not increase the sum of the diameters by 5 mm or more is coded as stable disease). To be assigned a status of stable disease, measurements must have met the stable disease criteria at least once after study entry at a minimum interval of 6 weeks.

With respect to non-target lesions, responses to therapy may include:

Complete Response (CR): Disappearance of all non-target lesions and normalization of tumor marker level. All lymph nodes must be non-pathological in size (<10 mm short axis). If tumor markers are initially above the upper normal limit, they must normalize for a patient to be considered in complete clinical response;

Non-CR/Non-PD: Persistence of one or more non-target lesion(s) and/or maintenance of tumor marker level above the normal limits; and

Progressive Disease (PD): Appearance of one or more new lesions and/or unequivocal progression of existing non-target lesions. Unequivocal progression should not normally trump target lesion status. It must be representative of overall disease status change, not a single lesion increase.

In exemplary outcomes, patients treated according to the methods disclosed herein may experience improvement in at least one sign of a cancer.

In one embodiment, the patient so treated exhibits CR, PR, or SD.

In another embodiment, the patient so treated experiences tumor shrinkage and/or decrease in growth rate, i.e., suppression of tumor growth. In yet another embodiment, one or more of the following can occur: the number of cancer cells is reduced; tumor size is reduced; cancer cell infiltration into peripheral organs is inhibited, retarded, slowed, or stopped; tumor metastasis is slowed or inhibited; tumor growth is inhibited; recurrence of tumor is prevented or delayed; or one or more of the symptoms associated with cancer is relieved to some extent.

In other embodiments, such improvement is measured by a reduction in the quantity and/or size of measurable tumor lesions. Measurable lesions are defined as those that can be accurately measured in at least one dimension (longest diameter is to be recorded) as >10 mm by either or both of CT scan (CT scan slice thickness no greater than 5 mm) and caliper measurement via clinical exam, or as >20 mm by chest X-ray. The size of non-target lesions, e.g., pathological lymph nodes, can also be measured for improvement. In one embodiment, lesions can be measured on chest x-rays or CT or MRI outputs.

In another embodiment, tumor progression is measured by assessing glycolic activity. For example, glycolic activity can be measured with positron emission tomography (PET). Fludeoxyglucose, also commonly called fluorodeoxyglucose and abbreviated [18F]FDG, 18F-FDG or FDG, is a radiopharmaceutical used in the medical imaging modality positron emission tomography (PET). The uptake of 18F-FDG by tissues is a marker for the tissue uptake of glucose, which in turn is closely correlated with certain types of tissue metabolism. After 18F-FDG is injected into a patient, a PET scanner can form two-dimensional or three-dimensional images of the distribution of 18F-FDG within the body.

In other embodiments, cytology or histology can be used to evaluate responsiveness to a therapy. The cytological confirmation of the neoplastic origin of any effusion that appears or worsens during treatment when the measurable tumor has met criteria for response or stable disease can be considered to differentiate between response or stable disease (an effusion may be a side effect of the treatment) and progressive disease.

### IX. Kits and Unit Dosage Forms

Further provided are kits containing a dendritic cell mobilizing agent (e.g., Flt3-L) and instructions for use according to the methods described herein. Kits typically include a packaged combination of reagents in predetermined amounts with instructions and a label indicating the intended use of the contents of the kit. The term label or instruction includes any writing, or recorded material supplied on or with the kit, or which otherwise accompanies the kit at any time during its manufacture, transport, sale or use. It can be in the form prescribed by a governmental agency regulating the manufacture, use or sale of pharmaceuticals or biological products, which notice reflects approval by the agency of the manufacture, use or sale for administration to a human or for veterinary use. The label or instruction can also encompass advertising leaflets and brochures, packaging materials, and audio or video instructions.

For example, in some embodiments, the kit contains the dendritic cell mobilizing agent in suitable containers and instructions for administration in accordance with the treatment regimens described herein. In some embodiments, the kit further comprises an additional immunoregulatory agent and/or an anti-cancer agent. In some embodiments, the dendritic cell mobilizing agent is provided in suitable containers as a dosage unit for administration. Suitable containers include, for example, bottles, vials, syringes, and test tubes. The containers may be formed from a variety of materials such as glass or plastic.

All references cited throughout this application, for example patent documents including issued or granted patents or equivalents; patent application publications; and non-patent literature documents or other source material; are hereby incorporated by reference herein in their entireties, as though individually incorporated by reference. Any sequence listing and sequence listing information is considered part of the disclosure herewith.

Those skilled in the art will recognize, or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

The following examples are merely illustrative and should not be construed as limiting the scope of this disclosure in any way as many variations and equivalents will become apparent to those skilled in the art upon reading the present disclosure.

### EXAMPLES

### Example 1: Treatment of Advanced Non-Small Cell Lung Cancer with Flt3 Ligand and Stereotactic Body Radiotherapy (SBRT)

Patients with advanced non-small cell lung cancer received daily subcutaneous injections of recombinant human Flt3 ligand (CDX-301; 75 μg/kg) for 5 days, beginning on the first day of stereotactic body radiotherapy (SBRT). In each case SBRT was delivered to a single pulmonary lesion. Depending on the size and location of the target lesion, the SBRT regimen was 34 Gy×1 (peripheral tumors smaller than 2 cm and not adjacent to the chest wall), 18 Gy×3 (peripheral tumors not eligible for 34 Gy×1), or 10 Gy×5 (central tumors).

The primary endpoint was progression-free survival at 4 months, defined using immune-related response criteria (irRC).

Secondary Outcome Measures were Dose Limiting Toxicities (DLTs): for the purposes of this study, a DLT was defined as any treatment-emergent grade 3-5 toxicity, scored using the Common Terminology Criteria for Adverse Events (CTCAE) version 4.0, and occurring within 30 days after treatment with SBRT in combination with FLT3 ligand therapy. Asymptomatic laboratory abnormalities (eg: leukocytosis) that did not require intervention were not counted as DLTs. For subjects who received more than one “cycle” of SBRT and FLT3 ligand, only adverse events that occurred after the first cycle were scored as potential DLTs.

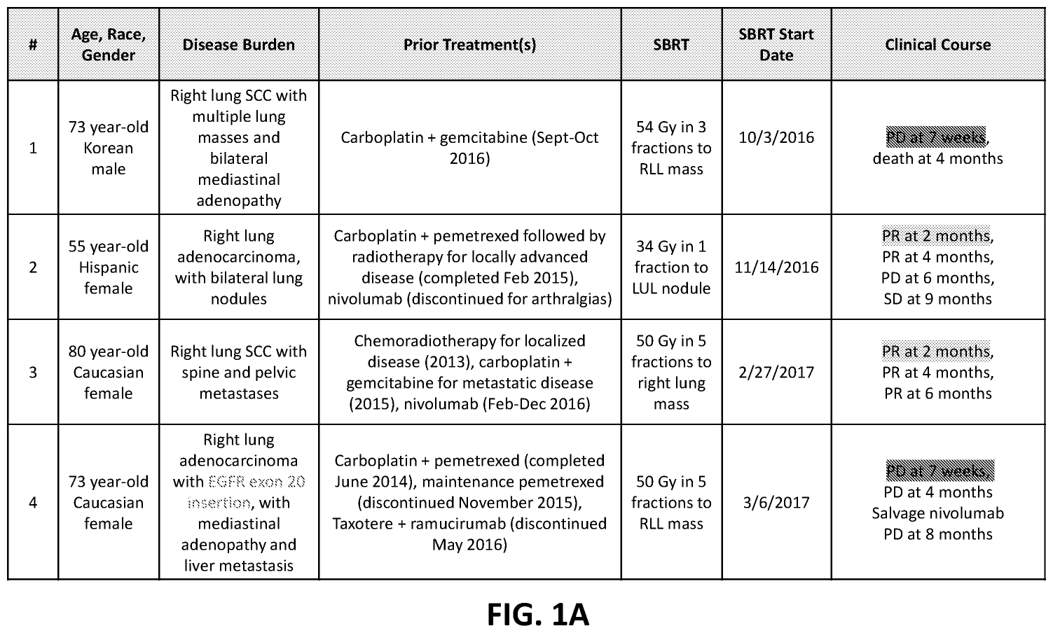

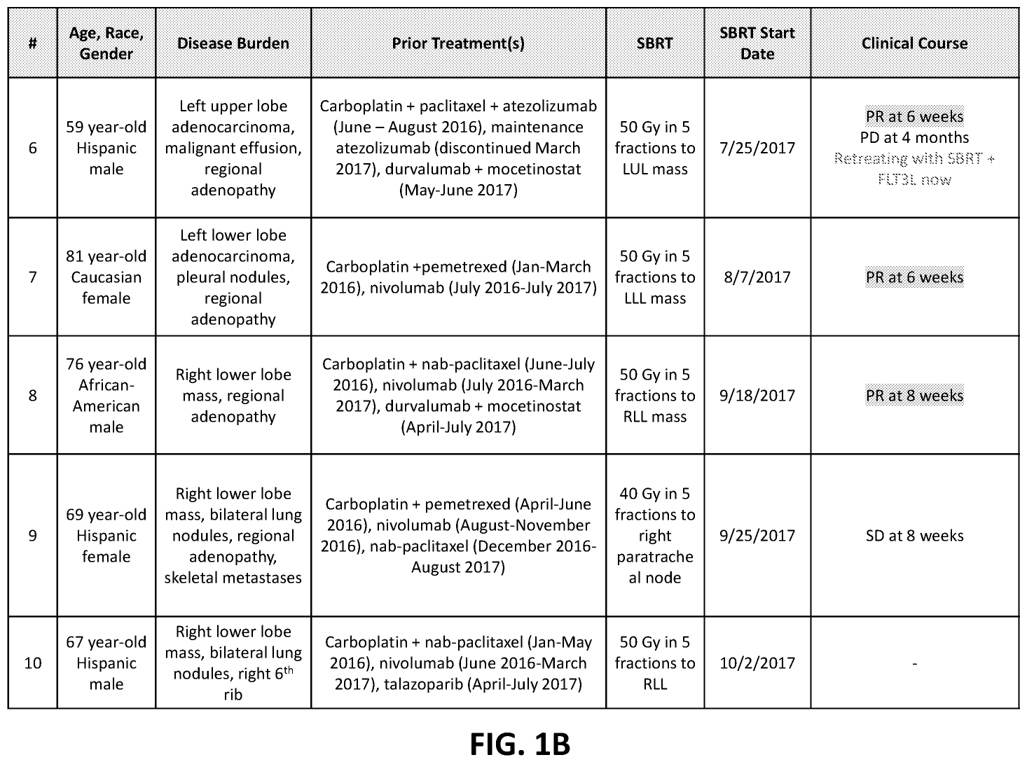

Preliminary results for the first 9 patients treated are shown in FIGS. 1A and 1B, in which PR denotes “Partial Response”, SD denotes “Stable Disease” and PD denotes “Progressive Disease”.

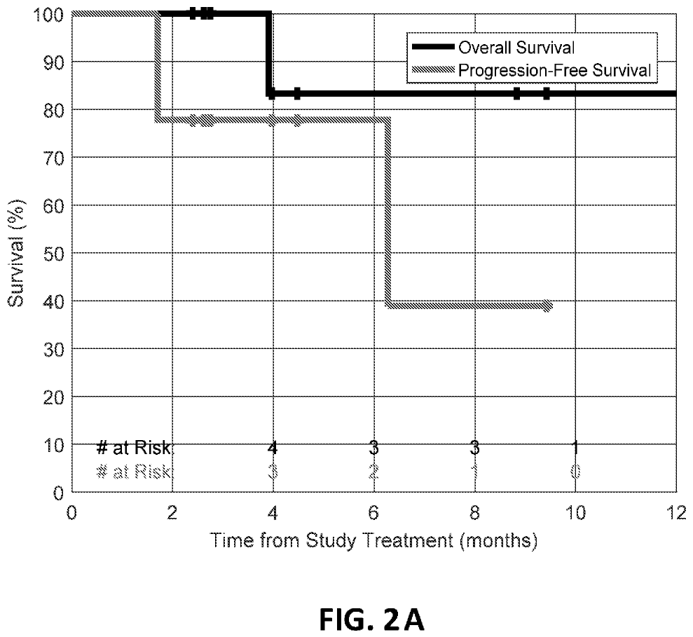

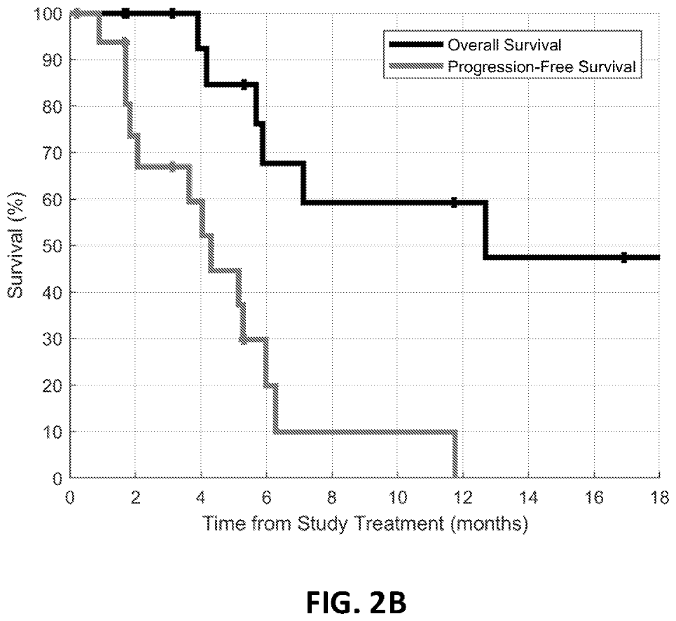

Overall survival and progression free survival are shown in FIGS. 2A and 2B.

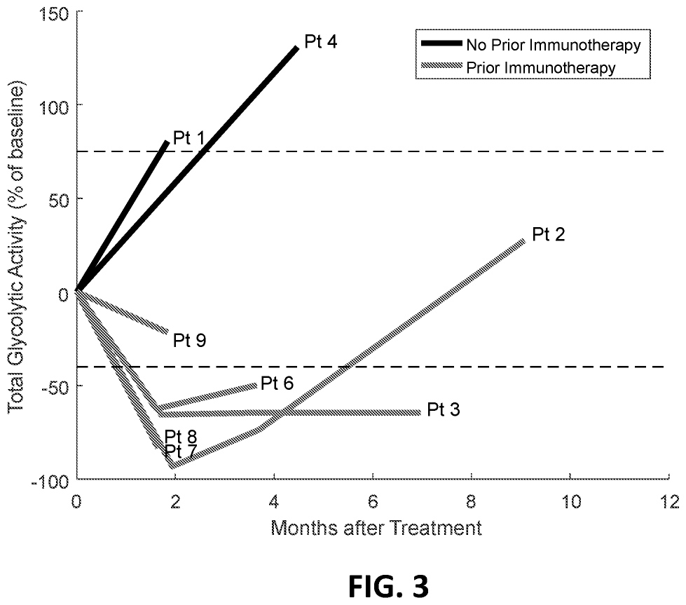

Total Glycolytic Activity (TGA) of non-SBRT treated lesions was measured by a blinded nuclear medicine specialist and is shown in FIG. 3. FIG. 3 shows the composite TGA of all lesions that were not treated with SBRT. Lesions treated with SBRT were excluded, so as to demonstrate ‘abscopal’ responses and not responses to SBRT. It can be seen that target lesions of patients who had received prior immunotherapy (checkpoint inhibitor therapy) had significantly lower TGA and several such patients exhibited partial responses.

SEQUENCE LISTING SUMMARY

| DESIGNATION | SEQUENCE | |
|---|---|---|
| Murine Flt3 | ATG ACA GTG CTG GCG CCA GCC TGG AGC CCA | SEQ ID NO: 1 |
| ligand cDNA | AAT TCC TCC CTG TTG CTG CTG TTG CTG CTG | |
| sequence | CTG AGT CCT TGC CTG CGG GGG ACA CCT GAC | |
| | TGT TAC TTC AGC CAC AGT CCC ATC TCC TCC | |
| | AAC TTC AAA GTG AAG TTT AGA GAG TTG ACT | |
| | GAC CAC CTG CTT AAA GAT TAC CCA GTC ACT | |
| | GTG GCC GTC AAT CTT CAG GAC GAG AAG CAC | |
| | TGC AAG GCC TTG TGG AGC CTC TTC CTA GCC | |
| | CAG CGC TGG ATA GAG CAA CTG AAG ACT GTG | |
| | GCA GGG TCT AAG ATG CAA ACG CTT CTG GAG | |
| | GAC GTC AAC ACC GAG ATA CAT TTT GTC ACC | |
| | TCA TGT ACC TTC CAG CCC CTA CCA GAA TGT | |
| | CTG CGA TTC GTC CAG ACC AAC ATC TCC CAC | |
| | CTC CTG AAG GAC ACC TGC ACA CAG CTG CTT | |
| | GCT CTG AAG CCC TGT ATC GGG AAG GCC TGC | |
| | CAG AAT TTC TCT CGG TGC CTG GAG GTG CAG | |
| | TGC CAG CCG GAC TCC TCC ACC CTG CTG CCC | |
| | CCA AGG AGT CCC ATA GCC CTA GAA GCC ACG | |
| | GAG CTC CCA GAG CCT CGG CCC AGG CAG CTG | |
| | TTG CTC CTG CTG CTG CTG CTG CCT CTC ACA | |
| | CTG GTG CTG CTG GCA GCC GCC TGG GGC CTT | |
| | CGC TGG CAA AGG GCA AGA AGG AGG GGG GAG | |
| | CTC CAC CCT GGG GTG CCC CTC CCC TCC CAT | |
| | CCC TAG | |
| Murine Flt3 | MTVLAPAWSPNSSLLLLLLLLSPCLRGTPDCYFSHSPIS | SEQ ID NO: 2 |
| ligand Amino | SNFKVKFRELTDHLLKDYPVTVAVNLQDEKHCKALWSLF | |
| Acid sequence | LAQRWIEQLKTVAGSKMQTLLEDVNTEIHFVTSCTFQPL | |
| | PECLRFVQTNISHLLKDTCTQLLALKPCIGKACQNFSRC | |
| | LEVQCQPDSSTLLPPRSPIALEATELPEPRPRQLLLLLL | |
| | LLPLTLVLLAAAWGLRWQRARRRGELHPGVPLPSHP | |
| Human Flt3 | ATG ACA GTG CTG GCG CCA GCC TGG AGC CCA | SEQ ID NO: 3 |
| ligand cDNA | ACA ACC TAT CTC CTC CTG CTG CTG CTG CTG | |
| sequence | AGC TCG GGA CTC AGT GGG ACC CAG GAC TGC | |
| | TCC TTC CAA CAC AGC CCC ATC TCC TCC GAC | |
| | TTC GCT GTC AAA ATC CGT GAG CTG TCT GAC | |
| | TAC CTG CTT CAA GAT TAC CCA GTC ACC GTG | |
| | GCC TCC AAC CTG CAG GAC GAG GAG CTC TGC | |
| | GGG GGC CTC TGG CGG CTG GTC CTG GCA CAG | |
| | CGC TGG ATG GAG CGG CTC AAG ACT GTC GCT | |
| | GGG TCC AAG ATG CAA GGC TTG CTG GAG CGC | |
| | GTG AAC ACG GAG ATA CAC TTT GTC ACC AAA | |
| | TGT GCC TTT CAG CCC CCC CCC AGC TGT CTT | |
| | CGC TTC GTC CAG ACC AAC ATC TCC CGC CTC | |
| | CTG CAG GAG ACC TCC GAG CAG CTG GTG GCG | |
| | CTG AAG CCC TGG ATC ACT CGC CAG AAC TTC | |
| | TCC CGG TGC CTG GAG CTG CAG TGT CAG CCC | |
| | GAC TCC TCA ACC CTG CCA CCC CCA TGG AGT | |
| | CCC CGG CCC CTG GAG GCC ACA GCC CCG ACA | |
| | GCC CCG CAG CCC CCT CTG CTC CTC CTA CTG | |
| | CTG CTG CCC GTG GGC CTC CTG CTG CTG GCC | |
| | GCT GCC TGG TGC CTG CAC TGG CAG AGG ACG | |
| | CGG CGG AGG ACA CCC CGC CCT GGG GAG CAG | |
| | GTG CCC CCC GTC CCC AGT CCC CAG GAC CTG | |
| | CTG CTT GTG GAG CAC TGA | |
| Human Flt3 | MTVLAPAWSPTTYLLLLLLLSSGLSGTQDCSFQHSPISS | SEQ ID NO: 4 |
| ligand Amino | DFAVKIRELSDYLLQDYPVTVASNLQDEELCGGLWRLVL | |
| Acid sequence | AQRWMERLKTVAGSKMQGLLERVNTEIHFVTKCAFQPPP | |
| | SCLRFVQTNISRLLQETSEQLVALKPWITRQNFSRCLEL | |
| | QCQPDSSTLPPPWSPRPLEATAPTAPQPPLLLLLLLPVG | |
| | LLLLAAAWCLHWQRTRRRTPRPGEQVPPVPSPQDLLLVE | |
| | H | |

What is claimed is:

1. A method for treating cancer in a subject by simultaneously or sequentially administering an immunoregulatory agent, a dendritic cell mobilizing agent, and radiation therapy.

2. A method for treating cancer in a subject who has become or been determined to be resistant to an immunoregulatory agent, by simultaneously or sequentially administering a dendritic cell mobilizing agent and radiation therapy.

3. The method of any one of the preceding claims, wherein the immunoregulatory agent is a checkpoint inhibitor.

4. The method of any one of the preceding claims, wherein the dendritic cell mobilizing agent is a Flt3 ligand or a nucleic acid coding therefor.

5. A method for treating cancer in a subject by simultaneously or sequentially administering an immune checkpoint inhibitor and a Flt3 ligand or a nucleic acid coding therefor.

6. A method for treating cancer in a subject who has become or been determined to be resistant to an immune checkpoint inhibitor, by administering a Flt3 ligand or a nucleic acid coding therefor.

7. The method of claim 5 or 6, wherein the method further comprises simultaneous or sequential administration of radiation therapy.

8. The method as claimed in any one of the preceding claims, wherein the Flt3 ligand is a soluble Flt3 ligand comprising the amino acid sequence of SEQ ID NO:4.

9. The method as claimed in any one of the preceding claims, wherein the immune checkpoint inhibitor includes an anti-PD1 antibody, an anti-PD-L1 antibody, or an anti-CTLA4 antibody.

10. The method as claimed in any one of the preceding claims, wherein the immune checkpoint inhibitor is nivolumab, pembrolizumab, atezolizumab, avelumab, durvalumab, tremelilumab or ipilimumab.

11. The method as claimed in any one of the preceding claims, wherein the radiation therapy is stereotactic body radiotherapy (SBRT).

12. The method as claimed in any one of the preceding claims wherein the cancer is leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

13. The method as claimed in any one of the preceding claims, wherein Flt3 ligand, or nucleic acid coding therefor, is administered for 1-10 days.

14. A method as claimed in any one of the preceding claims, wherein Flt3 ligand is administered at a dose of 10 to 200 ug/kg.

15. A method as claimed in claim 14, wherein Flt3 ligand is administered at a dose of 50 to 100 ug/kg.

16. A method as claimed in claim 15, wherein Flt3 ligand is administered at a dose of 75 ug/kg.

17. A method as claimed in any one of the preceding claims, wherein Flt3 ligand is administered for 1 to 10 days beginning between five days before or after the first day of radiotherapy.

18. A method as claimed in claim 17, wherein Flt3 ligand is administered for 1 to 10 days, beginning on the first day of radiotherapy.

19. A method as claimed in claim 18, wherein Flt3 ligand is administered for 5 days, beginning on the first day of radiotherapy.

20. A method as claimed in any one of the preceding claims, wherein the total radiation dose for a cycle of treatment is between 5 and 100 Gy.

21. A method as claimed in any one of the preceding claims, wherein the radiation dose for a cycle of treatment is between 20 and 50 Gy on one occasion, between 10 and 30 Gy on each of two to four occasions or between 5 and 20 Gy on each of 5 occasions.

22. A method as claimed in any one of the preceding claims, wherein the radiation dose for a cycle of treatment is 30 to 40 Gy on one occasion, 15 to 20 Gy on each of three occasions or 8 to 12 Gy on each of 5 occasions.

23. A method as claimed in any one of the preceding claims, comprising a step of administering a dendritic cell activating agent.

24. A method as claimed in claim 23, wherein the dendritic cell activating agent is CD40L, an anti-CD40 antibody, a TLR activator or a STING activator.

25. A method as claimed in any one of the preceding claims, wherein cycles of treatment are provided at intervals of 2 to 5 months.


SEQUENCE LISTING (4 sequences)

SEQ ID NO: 1 - DNA, 696 bp, Mus musculus
  misc_feature: Murine Flt3 ligand cDNA sequence
atgacagtgctggcgccagcctggagcccaaattcctccctgttgctgctgttgctgctgctgagtccttgcctgcgggggacacctgactgttacttcagccacagtcccatctcctccaacttcaaagtgaagtttagagagttgactgaccacctgcttaaagattacccagtcactgtggccgtcaatcttcaggacgagaagcactgcaaggccttgtggagcctcttcctagcccagcgctggatagagcaactgaagactgtggcagggtctaagatgcaaacgcttctggaggacgtcaacaccgagatacattttgtcacctcatgtaccttccagcccctaccagaatgtctgcgattcgtccagaccaacatctcccacctcctgaaggacacctgcacacagctgcttgctctgaagccctgtatcgggaaggcctgccagaatttctctcggtgcctggaggtgcagtgccagccggactcctccaccctgctgcccccaaggagtcccatagccctagaagccacggagctcccagagcctcggcccaggcagctgttgctcctgctgctgctgctgcctctcacactggtgctgctggcagccgcctggggccttcgctggcaaagggcaagaaggaggggggagctccaccctggggtgcccctcccctcccatccctag

SEQ ID NO: 2 - protein, 231 aa, Mus musculus
  misc_feature: Murine Flt3 ligand Amino Acid sequence
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLPLTLVLLAAAWGLRWQRARRRGELHPGVPLPSHP

SEQ ID NO: 3 - DNA, 708 bp, Homo sapiens
  misc_feature: Human Flt3 ligand cDNA sequence
atgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactga

SEQ ID NO: 4 - protein, 235 aa, Homo sapiens
  misc_feature: Human Flt3 ligand Amino Acid sequence
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH